(19) United States
(12) Patent Application Publication
(10) Pub. No.: US 2012/0046332 A1
(43) Pub. Date: Feb. 23, 2012

(54) COMPOSITIONS AND METHODS FOR TREATING CORNEAL HAZE

(75) Inventors: Guang Liang Jiang, Lake Forest, CA (US); Wha Bin Im, Irvine, CA (US); Larry A. Wheeler, Irvine, CA (US); Scott M. Whitcup, Laguna Hills, CA (US)

(73) Assignee: ALLERGAN, INC., Irvine, CA (US)

(21) Appl. No.: 13/211,715

(22) Filed: Aug. 17, 2011

Related U.S. Application Data
(60) Provisional application No. 61/374,439, filed on Aug. 17, 2010.

(51) Int. Cl. A61K 31/381; A61K 31/4025; A61P 27/02; A61K 31/4015

(52) U.S. Cl. 514/422; 514/443; 514/424

(57) ABSTRACT

Disclosed herein are compositions and methods for treating corneal haze. Compositions and methods of use comprise therapeutically effective amounts of compounds that agonize the EP2 and/or EP4 receptor. Administration of the disclosed compounds can prevent and treat corneal haze development.

### RELATED APPLICATIONS

This application claims the benefit of U.S. Provisional Application Ser. No. 61/374,439 filed on Aug. 17, 2010, disclosures of which are hereby incorporated in their entirety herein by reference.

### BACKGROUND

Corneal haze presents as a whitening of the normally clear cornea. This loss of transparency of the cornea can cause symptoms ranging from blurred vision to blindness. Corneal haze is common following photorefractive keratectomy (PRK), laser-assisted in-situ keratomileusis (LASIK), and laser epithelial keratomileusis (LASEK) to correct refractive errors. In these corrective procedures, the higher the level of myopic treatment, the more severe the resulting haze. Corneal haze also occurs in corneal infections and traumas, and in other ophthalmology surgeries, such as lens and cataract surgeries. Corneal haze is a significant problem as it reduces visual outcome, promotes regression of the obtained refraction, and may create glare and induce deterioration of neuron signal transmission.

Prevention of corneal haze is increasingly important as the number of refractive correction procedures and other ophthalmology surgeries increases world wide. Accordingly, compositions and methods that can prevent and/or treat corneal haze are highly desirable.

### BRIEF SUMMARY

The disclosure provides methods and compositions for treating and/or preventing corneal haze/opacity. In one embodiment, a method of treating corneal haze in an eye is provided. The method comprises administering a composition comprising a therapeutically effective amount of a compound selected from the group consisting of an EP2 agonist, and EP4 agonist, and a combination thereof, whereby the corneal haze is treated.

In another embodiment, provided are methods of treating corneal haze in an eye, by administering a composition comprising a therapeutically effective amount of a compound having a structure:

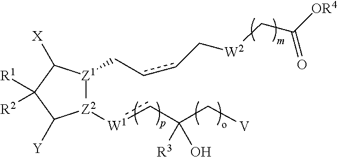

wherein each dashed line represents the presence or absence of a double bond;
R¹, R² and R³ are each independently selected from H or C₁-C₆ linear alkyl;
R⁴ is H, C₁-C₆ alkyl, C₁-C₆ alkenyl, a salt thereof, or an amine thereof;
X and Y are each independently selected from H, OH, ═O, Cl, Br, I, or CF₃;
Z¹ and Z² are each independently selected from CH or N;
W¹ and W² are each independently selected from CH, CH₂, aryl or substituted aryl, heteroaryl, substituted heteroaryl;
m is 0 to 4;
p is 0 or 1;
o is 0 to 4; and
V is CH₃, aryl, aryl or substituted aryl, heteroaryl, substituted heteroaryl,
wherein the administration treats the corneal haze.

In yet another embodiment, the disclosure provides a method of maintaining transparency of a cornea by administering a composition to the cornea, wherein the composition comprises a compound selected from the group consisting of an EP2 agonist, EP4 agonist, and a combination thereof.

In another embodiment, the disclosure provides a method of inhibiting transformation of a fibroblast to a myofibroblast in an eye, by administering a composition comprising a therapeutically effective amount of an EP2 agonist, an EP4 agonist or a combination thereof.

### BRIEF DESCRIPTION OF THE DRAWINGS

FIGS. 1A-D show immunohistochemical staining of human adult skin fibroblasts treated with vehicle (FIG. 1A), transforming growth factor beta-1 (TGF-β1; FIG. 1B), TGF-β1+ prostaglandin receptor subtype-4 (EP4) agonist (FIG. 1C), or TGF-β1+ prostaglandin receptor subtype 2 (EP2) agonist (FIG. 1D). TGF-β1 induced transformation of myofibroblasts, which stained green with anti-alpha-smooth muscle actin (α-SMA) immunocytochemistry.

FIG. 2 is a western blot showing the effect of EP2 and EP4 agonists on biomarker of myofibroblasts (α-SMA) in cultured adult skin fibroblasts. Parallel Western blots were conducted and total cell lysates were resolved by gel electrophoresis; anti-beta actin was used to quantify the total protein loading amounts.

### DETAILED DESCRIPTION

Certain terms as used in the specification are intended to refer to the following definitions, as detailed below. Where the definition of terms departs from the commonly used meaning of the term, Applicant intends to utilize the definitions provided below, unless specifically indicated.

“About” means plus or minus ten percent of the number, parameter or characteristic so qualified.

As used herein, “alkyl” refers to straight, branched chain or cyclic hydrocarbyl groups having from 1 up to about 100 carbon atoms. Whenever it appears herein, a numerical range, such as “1 to 4” or “C₁-C₄”, refers to each integer in the given range; e.g., “C₁-C₄ alkyl” means that an alkyl group may comprise only 1 carbon atom, 2 carbon atoms, 3 carbon atoms, Or 4 atoms. Although, the term “alkyl” also includes instances where no numerical range of carbon atoms is designated. “Substituted alkyl” refers to alkyl moieties bearing substituents typically selected from hydrogen, deuterium, alkyl, alkenyl, alkynyl, hydroxy, alkoxy, heterocyclic, aryl, heteroaryl, aryloxy, halogen, haloalkyl, cyano, nitro, amino, lower alkylamino, lower dialkylamino, amido, azido, acyl (—C(O)R⁶), alkoxymethyl, mercapto (—S—R⁶), sulfoxy (—S(O)—R⁶), sulfonyl (—S(O)₂—R⁶), sulfonamide (—S(O)₂N(R⁶)₂), carbonate (—OC(O)—O—R⁶), oxyacyl (—OC(O)—R⁶), carboxyl (—C(O)OH), ester (—C(O)OR⁶), carbamate (—OC(O)—N(R⁶)₂), wherein R⁶ is H or lower alkyl, lower alkenyl, lower alkynyl, aryl, heteroaryl, heterocycle, and the like. As used herein, “lower alkyl” refers to alkyl moieties having from 1 to about 4 or 1 to about 6 carbon atoms.

As used herein, “alkenyl” refers to straight, branched chain or cyclic hydrocarbyl groups having at least one carbon-carbon double bond, and having in the range of about 2 up to about 100 carbon atoms, and “substituted alkenyl” refers to alkenyl groups further bearing one or more substituents as set forth above. As used herein, “lower alkenyl” refers to alkenyl moieties having from 1 to about 6 carbon atoms.

As used herein, “alkynyl” refers to straight or branched chain hydrocarbyl groups having at least one carbon-carbon triple bond, and having in the range of about 2 up to about 100 carbon atoms, and “substituted alkynyl” refers to alkynyl groups further bearing one or more substituents as set forth above. As used herein, “lower alkynyl” refers to alkynyl moieties having from 2 to about 4 or 2 to about 6 carbon atoms.

As used herein, “cycloalkyl” refers to cyclic (i.e., ring-containing) alkyl moieties typically containing in the range of about 3 up to about 8 carbon atoms, and “substituted cycloalkyl” refers to cycloalkyl groups further bearing one or more substituents as set forth above.

As used herein, “aryl” refers to aromatic groups having in the range of 6 up to 14 carbon atoms and “substituted aryl” refers to aryl groups further bearing one or more substituents as set forth above.

As used herein, “heteroaryl” refers to aromatic moieties containing one or more heteroatoms (e.g., N, O, S, or the like) as part of the ring structure and having in the range of 5 up to 14 total atoms in the ring structure (i.e., carbon atoms and heteroatoms). “Substituted heteroaryl” refers to heteroaryl groups further bearing one or more substituents as set forth above.

As used herein, “heterocyclic” or “heterocycle” refers to non-aromatic cyclic (i.e., ring-containing) groups containing one or more heteroatoms (e.g., N, O, S, or the like) as part of the ring structure, and having in the range of 3 up to 14 carbon atoms and “substituted heterocyclic” or “substituted heterocycle” refers to heterocyclic groups or heterocycles further bearing one or more substituents as set forth above.

As used herein, “halogen” or “halide” refers to fluoride, chloride, bromide or iodide (F, Cl, Br, or I). The terms “fluoro”, “chloro”, “bromo”, and “iodo” may also be used when referring to halogenated substituents, for example, “trifluoromethyl.”

As used herein, “hydroxyalkyl” refers to alkyl-OH, such as hydroxymethyl, hydroxyethyl, and the like.

As used herein, “alkylacyl” refers to an alkyl ketone such as ethanone, propanone, and the like.

As used herein, “pharmaceutically acceptable salt” refers to any salt that retains the activity of the parent compound and does not impart any additional, deleterious or untoward effects on the subject to which it is administered and in the context in which it is administered compared to the parent compound. A pharmaceutically acceptable salt also refers to any salt which may form in vivo as a result of administration of an acid, another salt, or a prodrug which is converted into an acid or salt. Further, pharmaceutically acceptable salt refers to those salts which retain the biological effectiveness and properties of the free bases and which are obtained by reaction with inorganic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

Pharmaceutically acceptable salts of acidic functional groups may be derived from organic or inorganic bases. The salt may comprise a mono or polyvalent ion. Of particular interest are the inorganic ions, lithium, sodium, potassium, calcium, and magnesium. Organic salts may be made with amines, particularly ammonium salts such as mono-, di- and trialkyl amines or ethanol amines. Salts may also be formed with caffeine, tromethamine and similar molecules. Hydrochloric acid or some other pharmaceutically acceptable acid may form a salt with a compound that includes a basic group, such as an amine or a pyridine ring.

As used herein, the term “therapeutically effective amount” means the amount of the pharmaceutical composition that will elicit the biological or medical response of a subject in need thereof that is being sought by the researcher, veterinarian, medical doctor or other clinician.

The present disclosure provides compositions and methods for treating (i.e., preventing or reducing) corneal haze comprising an EP2 agonist, EP4 agonist, or a combination thereof. Methods of maintaining corneal transparency and inhibiting transformation of fibroblasts to myofibroblasts are disclosed as well.

Corneal haze is also referred to as cornea opacity, corneal clouding, corneal opacities, and corneal subepithelial haze. Corneal haze, such as after excimer laser photoablation, is due to stromal changes induced by the wound healing process. The generation of corneal myofibroblasts has recently been identified as the primary biological event responsible for the formation of corneal haze. Myofibroblasts are highly contractile cells with reduced transparency attributable to decreased intracellular crystalline production. In the healing process, TGF-β1 triggers the transformation of quiescent keratocytes into corneal fibroblasts and myofibroblasts, and stimulates the de-novo synthesis of extracellular matrix proteins.

It has been discovered that EP2 and EP4 agonists can prevent TGF-β1-induced morphological transformation of fibroblasts to myofibroblasts. Without wishing to be bound by any particular theory, it is thought that a reduction in the number of myofibroblasts, which are less transparent, reduces the development of opacity of the cornea. Therefore, administration of an EP2 and/or EP4 agonist can reduce the transformation of fibroblasts to myofibroblasts and therefore can prevent or reduce corneal haze formation.

In this regard, in one embodiment, a method is provided for inhibiting the transformation of a fibroblast to a myofibroblast by administering a therapeutically effective amount of an EP2 and/or EP4 agonist. The EP2 and/or EP4 agonist inhibits the transformation and further treats corneal haze formation.

In yet another embodiment, the disclosed compositions comprising a therapeutically effective amount of an EP2 and/or EP4 agonist can be administered to an eye to maintain corneal transparency.

Any EP2 and or EP4 agonist can be employed in the disclosed methods. That is, compounds selective for an EP2 receptor (i.e., Compounds I, II, and III), compounds selective for an EP4 receptor (i.e., Compounds IV, and V), and nonselective compounds that agonize both EP2 and EP4 receptors can be utilized in the claimed methods.

In certain embodiments, the methods disclosed herein comprise administering a composition comprising a therapeutically effective amount of a compound having a structure of formula I:

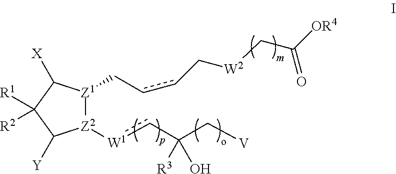

wherein each dashed line represents the presence or absence of a double bond;
R¹, R² and R³ are each independently selected from H or C₁-C₆ linear alkyl;
R⁴ is H, C₁-C₆ alkyl, C₁-C₆ alkenyl, a salt thereof, or an amine thereof;
X and Y are each independently selected from H, OH, ═O, Cl, Br, I, or CF₃;
Z¹ and Z² are each independently selected from CH or N;
W¹ and W² are each independently selected from CH, CH₂, aryl or substituted aryl, heteroaryl, substituted heteroaryl;
m is 0 to 4;
p is 0 or 1;
o is 0 to 4; and
V is CH₃, aryl, aryl or substituted aryl, heteroaryl, substituted heteroaryl.

In one embodiment, V is

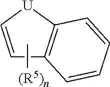

wherein R⁵ is halogen, C₁-C₆ alkyl, or C₁-C₆ alkenyl;
n is 0-7; and

### U is S or O.

In another embodiment, n is 1, U is S and R⁵ is Cl.

In one embodiment W² is thiophene.

In another embodiment, the compound has a structure

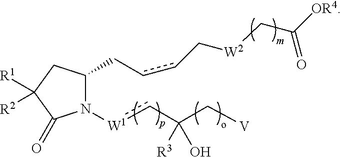

In yet another embodiment, the compound has a structure

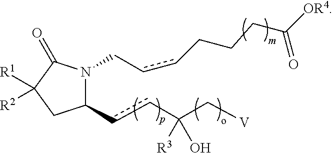

Further embodiments provide compounds with a structure

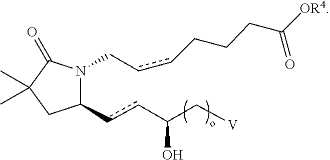

In certain embodiments, the composition comprises a therapeutically effective amount of Compound I:

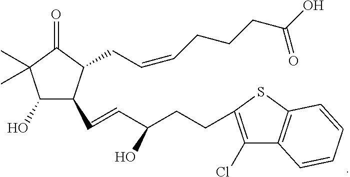

In another embodiment, the composition comprises a therapeutically effective amount of Compound II:

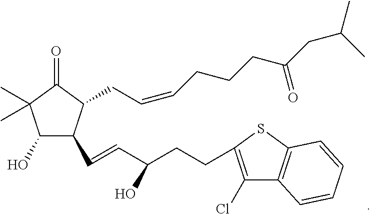

In another embodiment, the composition comprises a therapeutically effective amount of Compound III:

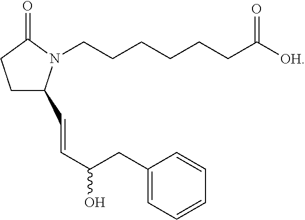

In another embodiment, the composition comprises a therapeutically effective amount of Compound IV:

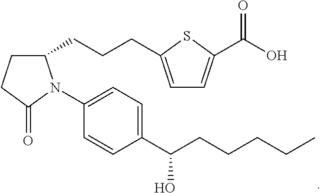

In another embodiment, the composition comprises a therapeutically effective amount of Compound V:

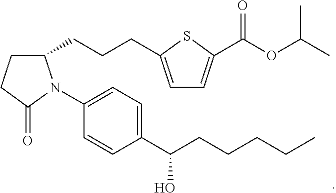

In certain embodiments, a combination of compounds can be employed. For instance, in one embodiment, 2 or more EP2 agonists are administered. In another embodiment, 2 or more EP4 agonists are administered. In yet another embodiment, an EP2 agonist and an EP4 agonist are administered. Any number and combination of compounds can be employed in accordance with the disclosed methods.

Methods of preparing the disclosed compounds and additional compounds suitable for use in the methods disclosed herein, can be found in, e.g., Donde, et el., 10,10-Dialkyl Prostanoic Acid Derivatives as Agents for Lowering Intraocular Pressure, U.S. Pat. No. 6,875,787; Donde, et el., 10,10-Dialkyl Prostanoic Acid Derivatives as Agents for Lowering Intraocular Pressure, U.S. Patent Publication 2004/0235958; Donde, et al., Treatment of Inflammatory Bowel Disease, U.S. Patent Publication 2005/0164992, each of which is hereby incorporated by reference in its entirety.

The disclosed compositions can be administered to an eye locally, that is, topically or intraocularly. Topical formulations include ointments, creams, gels, solutions, suspensions, etc. For instance, topical administration of a solution can be accomplished by administering one or more drops of a disclosed solution into an eye in need of treatment. Intraocular administration can be accomplished by placement of a biodegradable implant in an anterior location of an eye.

Pharmaceutical compositions comprising the disclosed compounds may be prepared by combining a therapeutically effective amount of at least one compound according to the present disclosure, or a pharmaceutically-acceptable salt thereof, as an active ingredient, with conventional ophthalmically acceptable pharmaceutical excipients, and by preparation of unit dosage forms suitable for topical ocular use. The therapeutically efficient amount typically is between about 0.0001 and about 5% (w/v), preferably about 0.001 to about 1.0% (w/v) in liquid formulations.

In certain embodiments, ophthalmic solutions can be prepared using a physiological saline solution as a major vehicle. In other embodiments, vehicles include, but are not limited to, polyvinyl alcohol, povidone, hydroxypropyl methyl cellulose, poloxamers, carboxymethyl cellulose, hydroxyethyl cellulose and purified water.

The pH of such ophthalmic solutions should preferably be maintained between about 4.5 to about 8.0 or about 6.5 to 7.2 with an appropriate buffer system. The formulations may also contain conventional, pharmaceutically acceptable preservatives, tonicity agents, and surfactants.

Preservatives suitable for use in ophthalmic solutions include, but are not limited to, benzalkonium chloride, chlorobutanol, thimerosal, phenylmercuric acetate and phenylmercuric nitrate. A surfactant can be, for example, Tween 80.

Tonicity adjustors may be added as needed or desired. Suitable tonicity adjustors include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. Accordingly, buffers include acetate buffers, citrate buffers, phosphate buffers and borate buffers. Acids or bases may be used to adjust the pH of these formulations as needed.

The compositions can further comprise one or more ophthalmically acceptable antioxidants. Suitable antioxidants include, but are not limited to, sodium metabisulfite, sodium thiosulfate, acetylcysteine, butylated hydroxyanisole and butylated hydroxytoluene.

Additional excipient components that may be included in the ophthalmic preparations are chelating agents. A non-limiting example of an acceptable chelating agent is edentate disodium, although other chelating agents may also be used in place or in conjunction with it.

In certain embodiments, the ingredients can be used in amounts indicated in Table 1.

TABLE 1

| Ingredient | Amount (% w/v) |
|---|---|
| Active agent | 0.005-5 |
| Preservative | 0-0.10 |
| Vehicle | 0-40 |
| Tonicity adjustor | 0-10 |
| Buffer | 0.01-1.0 |
| pH adjuster | q.s. 4.5-8.0 |
| Antioxidant | As needed |
| Surfactant | As needed |
| Purified Water | Up to 100% |

As mentioned above, in addition to or in lieu of topical administration, the compositions can be administered via intraocular implant. The implant can be placed at any appropriate ocular site for treating the cornea, typically in an anterior location. Such placement or insertion of an intraocular implant is well within the skill of the art.

The implant can vary according to the preferred drug release profile, the particular EP2 and/or EP4 agonist employed, the condition of the eye to be treated, and the medical history of the patient. An implant is suitable for insertion (or implantation) in an anterior ocular region or site (i.e., subcorneal insertion) if it has a size (length, width, depth) such that it can be inserted or implanted without causing excessive tissue damage and without unduly physically interfering with the existing vision of the patient into which the implant is implanted or inserted. The implants of the disclosure include at least one of the compounds disclosed herein dispersed within a biodegradable polymer. The production of such implants is well known in the art and any method for making an intraocular implant is contemplated herein.

The selection of a biodegradable polymer matrix to be employed will vary with the desired release kinetics, patient tolerance, and the like. Polymer characteristics that are considered include, but are not limited to, the biocompatibility and biodegradability at the site of implantation, compatibility with the active agent of interest, and processing temperatures. The biodegradable polymer matrix usually comprises at least about 10, at least about 20, at least about 30, at least about 40, at least about 50, at least about 60, at least about 70, at least about 80, or at least about 90 weight percent of the implant. In one variation, the biodegradable polymer matrix comprises about 40% by weight of the implant.

Biodegradable polymer matrices which may be employed include, but are not limited to, polymers made of monomers such as organic esters or ethers, which when degraded result in physiologically acceptable degradation products. Anhydrides, amides, orthoesters, or the like, by themselves or in combination with other monomers, may also be used. The polymers are generally condensation polymers. The polymers may be crosslinked or non-crosslinked. If crosslinked, they are usually not more than lightly crosslinked, and are less than 5% crosslinked, usually less than 1% crosslinked.

For the most part, in addition to carbon and hydrogen, the polymers will include oxygen and nitrogen, particularly oxygen. The oxygen may be present as oxy, e.g., hydroxy or ether, carbonyl, e.g., non-oxo-carbonyl, such as carboxylic acid ester, and the like. The nitrogen may be present as amide, cyano, and amino.

Polymers of hydroxyaliphatic carboxylic acids, either homo- or copolymers, and polysaccharides can be used. Included among the polyesters, are homo- or copolymers of D-lactic acid, L-lactic acid, racemic lactic acid, glycolic acid, caprolactone, and combinations thereof. Copolymers of glycolic and lactic acid are of particular interest, where the rate of biodegradation is controlled by the ratio of glycolic to lactic acid. The percent of each monomer in poly(lactic-co-glycolic)acid (PLGA) copolymer may be 0-100%, about 15-85%, about 25-75%, or about 35-65%. In a preferred variation, a 50/50 PLGA copolymer is used. In certain embodiments, a random copolymer of 50/50 PLGA is used.

Additional active agents can be utilized in conjunction with the compounds disclosed herein, in the same or a separate composition for topical or intraocular administration. Such additional active agents include, but are not limited to, ace-inhibitors, endogenous cytokines, agents that influence basement membrane, agents that influence the growth of endothelial cells, adrenergic agonists or blockers, cholinergic agonists or blockers, aldose reductase inhibitors, analgesics, anesthetics, antiallergics, anti-inflammatory agents, antihypertensives, pressors, antibacterials, antivirals, antifungals, antiprotozoals, anti-infectives, antitumor agents, antimetabolites, antiangiogenic agents, tyrosine kinase inhibitors, antibiotics, analgesics, antiallergic agents, antihelminthic agents, antiamebic agents, antifungal agents, anti-angiogenesis compounds anti-glaucoma agents, anti-neoplastics, antimetabolites, immunosuppressants, protease inhibitors, and various growth factors.

The disclosed compositions can be administered prior to, during, or after a corrective procedure such as PRK, LASIK, or LASEK. Administration after a procedure includes a dose once at the completion of the procedure, and/or in the hours, days, weeks, and months following the procedure. Administration can continue for a period of time such that corneal haze development can be prevented or reduced. Further, the compositions can be administered to only one or to both eyes, as needed.

Corneal haze is “treated” when the amount or severity of corneal haze that would typically develop in a patient similarly situated is reduced or prevented entirely. For instance, the degree of corneal haze that typically develops following a corrective procedure is proportional to the severity of the myopic condition being treated. Therefore, a patient with a severe myopic condition would be considered to be treated if that patient develops less (i.e., about 90%, 80%, 70%, 50%, 40%, 30%, 20%, or 10% less) corneal haze that the amount that typically develops in a patient similarly situated. Treatment therefore encompasses prevention or a reduction of corneal haze development following a corrective procedure.

The ophthalmic formulations can be packaged in forms suitable for metered application, such as in containers equipped with a dropper, to facilitate application to the eye. Containers suitable for drop wise application are usually made of suitable inert, non-toxic plastic material, and generally contain between about 0.5 and about 15 ml solution. One package may contain one or more unit doses.

The patient, as used herein, can be any mammal, typically a human. The human can be any age, gender or ethnicity.

### Example 1

### EP2 and EP4 agonists inhibit TGF-β1-induced myofibroblast transformation

Methods. Human fetal skin fibroblasts, human adult skin fibroblasts and adult keratinocytes were purchased from American Type Culture Collection (ATCC). Cells were cultured in high glucose Dulbecco's modified eagle's medium (DMEM) with 10% fetal bovine serum, 1% penicillin and streptomycin at 37° C. in the presence of 5% CO₂. To study EP2 and EP4 agonists mediated signal transduction, cells were seeded on 10-cm dishes. When reaching 90% confluency, cells were treated by an EP2 agonist (Compound III) and an EP4 agonist (Compound I) at 100 nM and 10 nM, respectively, or vehicle at indicated time points. Cell lysates were used for western blot analysis.

Adult skin keratinocytes were seeded into 6-well plates with pre-installed cover glasses. 80% confluent keratinocytes were treated with EP2 agonist Compound III at 100 nM or EP4 agonist Compound I at 10 nM for 2.5 hours with or without Akt inhibitor. BrdU was added at the last half hour of culture. Then the cells were stained by anti-BrdU immunocytochemistry and co-stained with 4′,6-diamidino-2-phenylindole (DAPI). BrdU-positive cells were quantified over DAPI stained cells in at least 5 fields under 200× magnification.

To perform the myofibroblast transformation assay, adult skin fibroblasts were cultured until 70% confluent, washed with phosphate buffered saline (PBS), and then starved in serum-free medium for 48 hours. Post starvation, cells were treated with TGF-β1 at 2 ng/ml alone or in combination with Compound V—100 nM, Compound II—10 nM or vehicle for 96 hrs in a fresh serum-free medium (TGF-beta1 from Sigma Aldrich, USA). Cell lysates were collected at the end of the study and Western blot was performed to monitor the expression of alpha-smooth muscle actin (α-SMA), the marker of myofibroblasts.

Cells were washed with cold PBS three times and lysed with a lysis buffer containing phosphatase inhibitors and proteinase inhibitors (Invitrogen, San Diego, Calif.). Protein level in lysates was quantified (bicinchoninic acid (BCA) protein assay kit, Pierce, USA). For Western blot, an equal amount of protein was applied at each lane, and subjected to electrophoresis. Gel-bound proteins after eletrophoretic resolution were transferred onto cellulose nitrate membranes, which were then hybridized with anti-pAKT, anti-pERK1/2, and HRP-conjugated second antibody. Fluorescence signals associated with target proteins were detected upon exposure to fluorescence-sensitive films (Cell signaling Technology, Inc., Berverly, Mass.). The membranes were then stripped with buffer provided by the vendor, and subjected to the same procedure with anti-AKT, anti-ERK1/2 and HRP-conjugated second antibody. For measuring the expression level of α-SMA, mouse anti-α-SMA antibody was used at 1:1000 dilution (Sigma Aldrich, USA). Beta-actin, a house-keeping gene, was also quantified on the same membrane as an internal control for loading amount.

To perform the myofibroblast transformation assay, adult skin fibroblasts were cultured until 70% confluent, washed with PBS, and then starved in serum-free medium for 48 hrs. Post starvation, cells were treated with TGF-beta1 at 2 ng/ml alone or in combination with Compound III—100 nM, Compound I—10 nM or vehicle for 96 hrs in a fresh serum-free medium (TGF-β1 from Sigma Aldrich, USA). Cell lysates were collected at the end of the study and Western blot was performed to monitor the expression of alpha-smooth muscle actin (α-SMA), the marker of myofibroblasts.

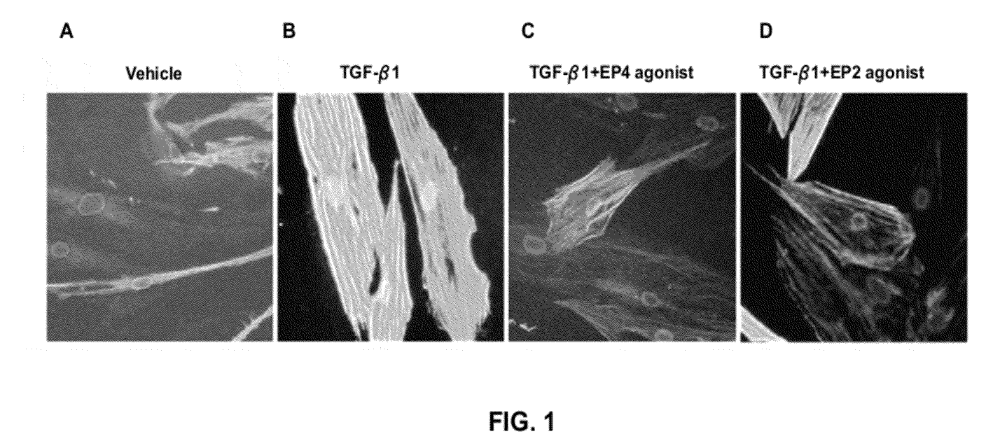

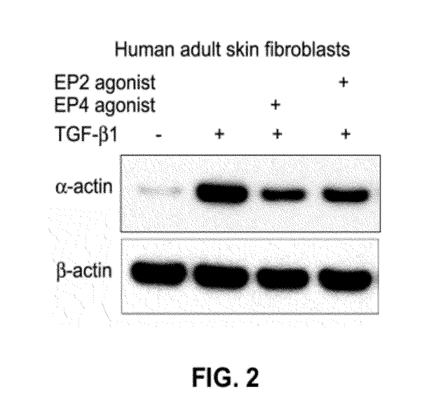

Normal skin fibroblasts are slim and spindle-shaped without detectable α-SMA (marker of myofibroblasts), 6 days post culturing in a serum-free medium (FIGS. 1-2). During the period, TGF-β1 treatment increased cell size by several folds, and induced cytoplasmic α-SMA positive stress fibers, a typical biomarker for myofibroblasts. Co-treatments of TGF-β1 with an EP4 or EP2 agonist significantly reduced α-SMA positive stress fibers, but not cell size. The results from Western blots were consistent with the morphological changes in that TGF-β1 markedly increased α-SMA expression from the barely detectable basal level, and EP2 agonist treatment reduced the α-SMA expression by approximately 50%, and EP4 agonist by nearly 75%.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, any references made to patents and printed publications throughout this specification are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed:

1. A method of treating corneal haze in an eye, by administering a composition comprising a therapeutically effective amount of a compound selected from the group consisting of an EP2 agonist, and EP4 agonist, and a combination thereof, whereby the corneal haze is treated.

2. The method of claim 1, wherein the corneal haze is caused by photorefractive keratectomy (PRK), laser-assisted in-situ keratomileusis (LASIK), or laser epithelial keratomileusis (LASEK).

3. The method of claim 1, wherein the compound is an EP2 agonist.

4. The method of claim 1, wherein the compound is an EP4 agonist.

5. The method of claim 1, wherein the compound is administered locally by eye drop, ointment, cream, or intraocular implant.

6. A method of treating a corneal haze in an eye, by administering a composition comprising a therapeutically effective amount of a compound having a structure: 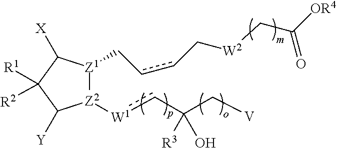
wherein each dashed line represents the presence or absence of a double bond;
R¹, R² and R³ are each independently selected from H or C₁-C₆ linear alkyl;
R⁴ is H, C₁-C₆ alkyl, C₁-C₆ alkenyl, a salt thereof, or an amine thereof;
X and Y are each independently selected from H, OH, ═O, Cl, Br, I, or CF₃;
Z¹ and Z² are each independently selected from CH or N;
W¹ and W² are each independently selected from CH, CH₂, aryl or substituted aryl, heteroaryl, substituted heteroaryl;
m is 0 to 4;
p is 0 or 1;
o is 0 to 4; and
V is CH₃, aryl, aryl or substituted aryl, heteroaryl, substituted heteroaryl.
wherein the administration treats the corneal haze.

7. The method of claim 6, wherein V is 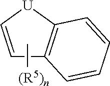
wherein R⁵ is halogen, C₁-C₆ alkyl, or C₁-C₆ alkenyl;
n is 0-7; and
U is S or O.

8. The method of claim 6, wherein n is 1, U is S and R⁵ is Cl.

9. The method of claim 6, wherein W² is thiophene.

10. The method of claim 6, wherein the compound has the structure:

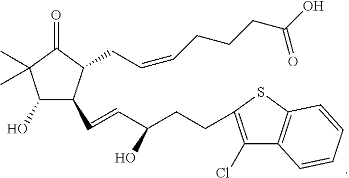

11. The method of claim 6, wherein the compound has the structure:

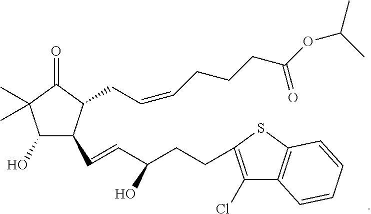

12. The method of claim 6, wherein the compound has the structure:

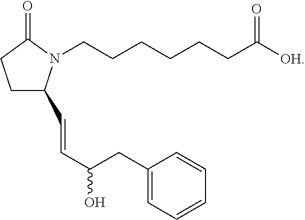

13. The method of claim 6, wherein the compound has the structure:

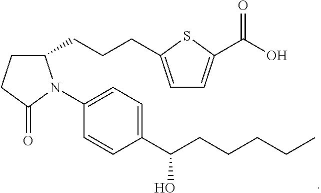

14. The method of claim 6, wherein the compound has the structure:

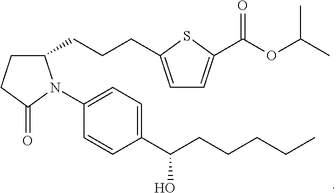

15. A method of maintaining transparency of a cornea by administering a composition to said cornea, wherein said composition comprises a compound selected from the group consisting of an EP2 agonist, EP4 agonist, and a combination thereof.

16. The method of claim 15, wherein the composition is administered prior to, during or after a procedure selected from PRK, LASEK, or LASIK.

17. The method of claim 15, wherein the compound has a structure:

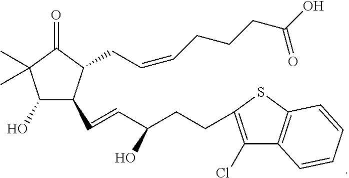

18. The method of claim 15, wherein the compound has a structure:

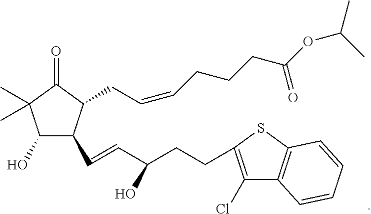

19. The method of claim 15, wherein the compound has a structure:

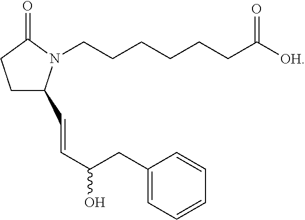

20. The method of claim 15, wherein the compound has a structure:

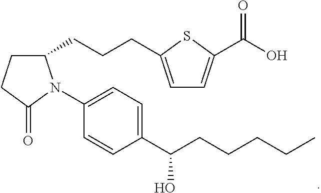

21. The method of claim 15, wherein the compound has a structure:

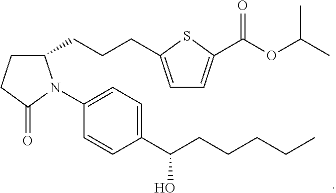

22. A method of inhibiting transformation of a fibroblast to a myofibroblast in an eye, by administering a composition comprising a therapeutically effective amount of an EP2 agonist, an EP4 agonist or a combination thereof, wherein the administration inhibits transformation of a fibroblast to a myofibroblast.

23. The method of claim 22, wherein by inhibiting transformation of the fibroblast to the myofibroblast, corneal haze is prevented or reduced.

23. The method of claim 22, wherein by inhibiting transformation of the fibroblast to the myofibroblast corneal transparency is maintained.

24. The method of claim 22, wherein the composition is administered prior to, during, or after a corrective procedure.

25. The method of claim 24, wherein the corrective procedure is PRK, LASIK, or LASEK.
